(19) United States
(12) Patent Application Publication
(10) Pub. No.: US 2011/0104492 A1
(43) Pub. Date: May 5, 2011

(54) HIGHLY EFFICIENT GAS PHASE METHOD FOR MODIFICATION AND FUNCTIONALIZATION OF CARBON NANOFIBRES WITH NITRIC ACID VAPOUR

(75) Inventors: Martin Muhler, Bochum (DE); Wei Xia, Bochum (DE)

(73) Assignee: Bayer Material Science AG, Leverkusen (DE)

(21) Appl. No.: 13/002,396

(22) PCT Filed: Jun. 27, 2009
(86) PCT No.: PCT/EP2009/004664

§ 371 Date: Jan. 3, 2011

(30) Foreign Application Priority Data
Jul. 3, 2008 (DE) 10 2008 031 579.6

(51) Int. Cl. D02G 3/02; C07D 311/02; B82Y 99/00; B82Y 40/00

(52) U.S. Cl. 428/375; 549/232; 977/762; 977/896

(57) ABSTRACT

The present invention relates to a method for the functionalization of carbon fibres using the vapour of nitric acid, carbon fibres thus modified and use thereof.

The present invention relates to a method for the functionalisation of carbon fibres with nitric acid vapour, carbon fibres modified in this way and the use thereof.

According to the prior art carbon nanofibres are understood to be mainly cylindrical carbon tubes with a diameter of between 3 and 100 nm and a length that is a multiple of the diameter. These tubes consist of one or more layers of oriented carbon atoms and have a core of a differing morphology. These carbon nanofibres are also known as carbon fibrils or hollow carbon fibres, for example.

Carbon nanofibres have long been known in the specialist literature. Although Iijima (publication: S. Iijima, Nature 354, 56-58, 1991) is generally described as the discoverer of nanotubes, these materials—especially fibrous graphite materials with multiple graphite layers—have been known since the 1970s or early 1980s. Tates and Baker (GB 1469930A1, 1977 and EP 56004 A2) were the first to describe the deposition of very fine fibrous carbon from the catalytic breakdown of hydrocarbons. However, the carbon filaments produced from short-chain hydrocarbons were not characterised in any further detail with regard to their diameter.

Conventional structures of these carbon nanofibres are those of the cylinder type. Within the cylindrical structures a distinction is made between single-walled monocarbon nanotubes and multi-walled cylindrical carbon nanotubes. Common methods for their manufacture include for example arc discharge, laser ablation, chemical vapour deposition (CVD) and catalytic chemical vapour deposition (CCVD).

The use of the arc discharge method to form carbon fibres which consist of two or more graphene layers and are rolled up into a seamless cylinder and nested inside one another is known from Iijima, Nature 354, 1991, 56-8. Depending on the rolling vector, chiral and achiral arrangements of the carbon atoms in relation to the longitudinal axis of the carbon fibres are possible.

Carbon fibre structures in which a single continuous graphene layer (scroll type) or discontinuous graphene layer (onion type) forms the basis for the structure of the nanotubes were first described by Bacon et al., J. Appl. Phys. 34, 1960, 283-90. The structure is known as the scroll type. Corresponding structures were subsequently also found by Zhou et al., Science, 263, 1994, 1744-47, and by Lavin et al., Carbon 40, 2002, 1123-30.

Owing to the inert and hydrophobic properties of carbon nanofibres, surface modification and functionalisation is essential for their use, particularly in catalysis (Toebes, M. L. et al., J. Catal. 214:78-87 (2003); de Jong K. P., Geus J. W., Catal. Rev.-Sci. Eng. 42:481-510 (2000); Serp P. et al., Appl. Catal. A 253:337-58 (2003); Nhut, J. M. et al., Appl. Catal. A 254:345-63 (2003)). One of the most frequently used methods of surface modification is the production of oxygen-containing functional groups by means of partial oxidation. On the one hand oxidation makes the carbon nanofibres hydrophilic, as a result of which an aqueous catalyst preparation is possible because of the improved wetting properties. On the other hand the oxygen-containing functional groups produced on the surface can serve as anchor points for catalyst precursor complexes. A key role here is ascribed to carboxyl groups (Boehm, H. P., Carbon 32:759:69 (1994)).

Many methods for the treatment of carbon nanofibres have been described in the literature. These include oxygen (Morishita, K., Takarada T., Carbon 35:977-81 (1997); Ajayan, P. M. et al., Nature 362:522-5 (1993); Ebbesen, T. W. et al., Nature 367:519-9 (1997)), ozone (Byl, O. et al., Langmuir 21:4200-4 (2005)), carbon dioxide (Tsang, S. C. et al., Nature 262:520-2 (1993); Seo, K. et al., J. Am. Chem. Soc. 125:13946-7 (2003)), water (Xia, W. et al., Mater 19:3648-52 (2007)), hydrogen peroxide (Xu, C. et al., Adv. Engineering Mater 8:73-77 (2006)) and plasma treatment (Bubert, H. et al., Anal. Bioanal. Chem. 374:1237-41 (2002)) as well as nitric acid treatment, the most frequently used of all (Lakshminarayanan, P. V. et al., Carbon 42:2433-42 (2004); Darmstadt, H. et al., Carbon 36:1183-90 (1998); Darmstadt, H. et al., Carbon 35:1581-5 (1997)). Nitrogen dioxide is used for processing traditional carbon materials such as for example amorphous carbon or carbon black (Jacquot, F. et al., 40:335-43 (2002); Jeguirim, M. et al., Fuel 84:1949-56 (2005)). One aim of these treatments can also be to clean, shred and open up the carbon nanofibres (Liu, J. et al., 280:1253-6 (1998)).

Only highly oxidising agents such as for example nitric acid or a mixture of nitric acid and sulfuric acid under aggressive reaction conditions can be used effectively for producing oxygen-containing functional groups, above all if a large amount of carboxyl groups is required (Toebes, M. L. et al., Carbon 42:307-15; Ros, T. G. et al., 8:1151-62 (2002)). However, this oxidation with corrosive acids in the liquid phase frequently gives rise to structural damage to the carbon nanofibres (Ros, T. G. et al., 8:1151-62 (2002); Zhang, J. et al., J. Phys. Chem. B 107:3712-8 (2003)), at least part of which is caused by mechanical stress due to refluxing and stirring. Furthermore, separating the treated carbon nanofibres from the acid is difficult, above all for carbon nanofibres of small diameter. Separation is normally carried out by filtration, causing a substantial amount of carbon nanofibres to be lost, however. In addition, the subsequent drying process frequently leads to agglomeration of the carbon nanofibres, and this has an influence on their usability.

Gas-phase treatment appears to be an attractive alternative for avoiding these problems. However, conventional gas-phase treatments with air, ozone, oxygen or plasma are usually less effective than treatment with nitric acid (Ros, T. G. et al., 8:1151-62 (2002)). In WO 06/135439 a maximum surface concentration of oxygen of 0.069 measured by XPS is obtained with the various oxidation methods used. It is also known that more carbonyl groups than carboxyl groups are formed with these methods because of the lack of water, meaning that the carbon nanofibres are functionalised less efficiently.

Oxidative treatment with corrosive acids in aqueous solution is currently the most effective method. The biggest disadvantages are as follows:

1. Mechanical stress, triggered by stirring and refluxing, is at least partly responsible for structural damage to the carbon nanofibres.

2. Separation by filtration of the acid-treated carbon nanofibres, particularly of small-diameter nanofibres, is associated with high losses.

3. The subsequent drying process also leads to agglomeration of the carbon nanofibres, reducing their usability.

Gas-phase methods are an attractive alternative to the conventional treatment methods as they avoid the aforementioned problems. However, conventional gas-phase treatments (ozone, air and plasma, etc.) are less effective as compared with treatment with nitric acid. It is also known that the lack of water means that carbonyl groups are preferentially formed to date, with carboxyl groups being less preferentially formed.

US 04/0253374 describes a method for cleaning and reinforcing carbon nanofibres with a pretreated dilute aqueous nitric acid solution and using helium as the carrier gas in a fluidised-bed reactor at temperatures of 400° C., in which nitro groups form at the surface. The disadvantage of this method is the use of large amounts of helium, which is necessary to hold the carbon nanofibre agglomerates in suspension, and the dust formed by the rubbing together of the carbon particles, which is carried out with the carrier gas.

WO 02/45812 A2 describes a cleaning method for carbon nanofibres in which the vapour is condensed before the fibres are treated, as a result of which the fibres have to be filtered.

The object of the present invention is therefore to provide a gas-phase method which is as simple as possible yet highly efficient and which allows modification and functionalisation of carbon fibres without structural and morphological changes.

In a first embodiment the object underlying the invention is achieved by means of a method for the functionalisation of carbon fibres wherein

a) carbon fibres 1 are placed in a reactor 2, which has an inlet 3 and an outlet 4,

b) the reactor 2 is heated to a temperature in a range from 125 to 500° C.,

c) vapour from nitric acid 5 is passed through the reactor 2, and

d) the treated carbon fibres are then dried.

“Nitric acid” within the meaning of the invention does not exclude the possibility of its being diluted with water or used in combination with sulfuric acid, for example.

A simple yet highly effective method for the functionalisation of carbon fibres by treatment with nitric acid vapour is therefore provided which avoids the problematic separation by filtration. In comparison to conventional wet HNO₃ treatment a significantly larger amount of oxygen species can be detected on the surface by means of X-ray photoelectron spectroscopy (XPS). The treatment does not impair the morphology or the degree of agglomeration.

A new gas-phase method for the oxidation and functionalisation of carbon nanofibres is therefore provided. Treatment with nitric acid vapour proves to be a more effective method of producing oxygen-containing functional groups on carbon nanofibre surfaces, for example, as compared with conventional methods with liquid nitric acid, wherein the morphology and the degree of agglomeration are not impaired and the treatment temperature can be freely selected. In addition, the use of HNO₃ gas-phase treatment is more advantageous because it avoids filtration, washing and drying steps.

Carbon nanofibres are advantageously used as carbon fibres, in particular those having an external diameter in a range from 3 to 500 nm. The diameter can be determined for example using transmission electron microscopy (TEM). If carbon fibres with a diameter below the preferred range are used, there is a possibility of the carbon fibres being destroyed during treatment or at least of their mechanical properties being severely compromised. If carbon fibres with an external diameter above the preferred range are used, the specific BET surface area can be too small for certain applications, such as catalysis for example.

Carbon nanofibres within the meaning of the invention are all single-walled or multi-walled carbon nanotubes of the cylinder or scroll type or having an onion-like structure. Multi-walled carbon nanotubes of the cylinder or scroll type or mixtures thereof are preferably used. Carbon nanofibres having a ratio of length to external diameter of greater than 5, preferably greater than 100, are particularly preferably used.

The carbon nanofibres are particularly preferably used in the form of agglomerates, wherein the agglomerates have in particular an average diameter in the range from 0.05 to 5 mm, preferably 0.1 to 2 mm, particularly preferably 0.2 to 1 mm.

By preference the carbon nanofibres to be used substantially have an average diameter of 3 to 100 nm, particularly preferably 5 to 80 nm, particularly preferably 6 to 60 nm.

Unlike the known CNTs of the scroll type mentioned at the start, which have only one continuous or discontinuous graphene layer, CNT structures have also been found by the applicant which consist of several graphene layers stacked together and rolled up (multi-scroll type). These carbon nanotubes and carbon nanotube agglomerates formed therefrom are provided for example by the as yet unpublished German patent application with the official filing number 102007044031.8. Its content with regard to CNTs and their manufacture is hereby included in the disclosure of this application. The way in which this CNT structure relates to the carbon nanotubes of the simple scroll type is comparable to the way in which the structure of multi-walled cylindrical monocarbon nanotubes (cylindrical MWNT) relates to the structure of single-walled cylindrical carbon nanotubes (cylindrical SWNT).

In contrast to the onion-type structures, when viewed in cross-section the individual graphene or graphite layers in these carbon nanofibres clearly run continuously from the centre of the CNTs to the outer edge without interruption. This can allow a better and faster intercalation of other materials in the tube skeleton, for example, as there are more open edges available as entry zones for the intercalates as compared with CNTs having a simple scroll structure (Carbon 34, 1996, 1301-3) or CNTs having an onion-type structure (Science 263, 1994, 1744-7).

The currently known methods for producing carbon nanotubes include the arc discharge, laser ablation and catalytic methods. In many of these methods carbon black, amorphous carbon and large-diameter fibres are formed as by-products. In the catalytic methods a distinction can be made between deposition of supported catalyst particles and deposition of metal centres formed in situ with diameters in the nanometre range (known as flow methods). For the production by catalytic deposition of carbon from hydrocarbons that are in gaseous form under the reaction conditions (referred to below as CCVD: catalytic carbon vapour deposition), acetylene, methane, ethane, ethylene, butane, butene, butadiene, benzene and other carbon-containing reactants are mentioned as possible carbon donors. CNTs obtainable by catalytic methods are therefore preferably used.

The catalysts generally contain metals, metal oxides or degradable or reducible metal components. Fe, Mo, Ni, V, Mn, Sn, Co, Cu and other subgroup elements, for example, are cited in the prior art as metals for the catalyst. Although the individual metals mostly have a tendency to support the formation of carbon nanotubes, high yields and small proportions of amorphous carbons are advantageously obtained according to the prior art with metal catalysts based on a combination of the aforementioned metals. Consequently the use of CNTs obtainable using mixed catalysts is preferred. Particularly advantageous catalyst systems for producing CNTs are based on combinations of metals or metal compounds containing two or more elements from the series Fe, Co, Mn, Mo and Ni.

Experience shows that the formation of carbon nanotubes and the properties of the tubes that are formed have a complex dependency on the metal component or combination of several metal components used as catalyst, on the catalyst support material optionally used and on the interaction between catalyst and support, the reactant gas and partial pressure, an admixture of hydrogen or other gases, the reaction temperature and the dwell time and the reactor used. A method that is particularly preferably used to produce carbon nanotubes is known from WO 2006/050903 A2.

In the various methods mentioned thus far using a variety of catalyst systems, carbon nanotubes of differing structures are produced which can largely be removed from the process as carbon nanotube powders.

Further carbon nanofibres that are preferably suitable for the invention are obtained by methods which are described in principle in the references below:

The production of carbon nanotubes with diameters of less than 100 nm is described for the first time in EP 205 556 B1. Light (i.e. short- and medium-chain aliphatic or mono- or binuclear aromatic) hydrocarbons and an iron-based catalyst are used for production here, on which carbon carrier compounds break down at a temperature above 800 to 900° C.

WO86/03455A1 describes the production of carbon filaments which have a cylindrical structure with a constant diameter of 3.5 to 70 nm, an aspect ratio (ratio of length to diameter) of greater than 100 and a core region. These fibrils consist of many continuous layers of oriented carbon atoms which are arranged concentrically around the cylindrical axis of the fibrils. These cylindrical nanotubes were produced by a CVD process from carbon-containing compounds by means of a metal-containing particle at a temperature of between 850° C. and 1200° C.

Another method for the production of a catalyst which is suitable for producing conventional carbon nanotubes with a cylindrical structure has become known from WO2007/093337A2. Using this catalyst in a fixed bed produces elevated yields of cylindrical carbon nanotubes with a diameter in the range from 5 to 30 nm.

A completely different way of producing cylindrical carbon nanofibres was described by Oberlin, Endo and Koyam (Carbon 14, 1976, 133). Here aromatic hydrocarbons such as benzene for example are reacted on a metal catalyst. The carbon tubes that are formed have a well-defined graphite hollow core with approximately the diameter of the catalyst particle, on which there is further less graphitically oriented carbon. The entire tube can be graphitised by treatment at high temperature (2500° C. to 3000° C.).

Most of the aforementioned methods (arc discharge, spray pyrolysis or CVD) are used today to produce carbon nanotubes. The production of single-walled cylindrical carbon nanotubes is very complex in terms of the apparatus involved, however, and with the known methods proceeds at a very slow rate of formation and often also with many secondary reactions which lead to a high proportion of undesired impurities, meaning that the yield from such methods is comparatively low. Even today the production of such carbon nanotubes is thus extremely technically complex, and they are therefore mostly used in small amounts for highly specialised applications. Their use is conceivable for the invention, however, but less preferable than the use of multi-walled CNTs of the cylinder or scroll type.

The production of multi-walled carbon nanotubes in the form of nested seamless cylindrical nanotubes or in the form of the scroll or onion structures described above takes place commercially today in relatively large volumes, mostly using catalytic methods. These methods usually demonstrate a higher yield than the aforementioned arc discharge and other methods and are typically performed today on the kilogram scale (a few hundred kg per day worldwide). The MW carbon nanotubes produced in this way are generally considerably less expensive than the single-walled nanotubes and for that reason are used for example as a performance-boosting additive in other materials.

For that reason carbon fibres having a BET surface area in a range from 10 to 500 m²/g, in particular in a range from 20 to 200 m²/g, are preferably also used. The BET specific surface area can be determined for example using a Porotec Sorptomatic 1990 in accordance with DIN 66131. If carbon fibres having a BET surface area below the preferred range are used, this can mean—as already indicated—that the carbon fibres are no longer suitable for certain applications, such as catalysis for example. If carbon fibres having a BET surface area above the preferred range are used, this can mean that the carbon fibres are too severely attacked or even destroyed during the treatment with nitric acid vapour.

In the method according to the invention a condenser 6 is preferably provided after the reactor outlet 4, the condenser outlet 7 for the condensate being connected via a return line 8 to a storage vessel 9 for the nitric acid 5. This can prevent condensed nitric acid in the liquid state from wetting the carbon fibres present in the reactor. In particular, treatment in the vapour phase of nitric acid allows the surface of carbon fibres to be modified with oxygen substantially better than in the liquid phase.

A glass flask which in particular is heated with an oil bath 10 is preferably used as the storage vessel 9 for the nitric acid. This storage vessel 9 is advantageously positioned below the reactor 2. In this way the vapour from the nitric acid, when it is heated in the glass flask by the oil bath, can come into contact with the carbon fibres through the reactor inlet. The reactor is therefore preferably positioned vertically, with the inlet for the nitric acid vapour positioned below the carbon fibres and the outlet positioned above the carbon fibres. The vapour can thus flow through the reactor and through the reactor outlet into the condenser, where the nitric acid is then condensed and returned to the storage vessel. The reactor 2 is heated by means of a heater 11, for example.

After step (b) the reactor is left at this temperature for a period in the range from 3 to 20 hours, in particular in a range from 5 to 15 hours. If a shorter time is allowed, the surface modification will be too slight. If this preferred range is exceeded, no further improvement in the surface modification will be seen. In particular the temperature for the treatment period is set to a temperature below 250° C. and independently thereof to a temperature above 150° C. These temperatures have proved to be particularly suitable for the surface modification of carbon fibres with oxygen.

Step (c), the drying stage, is preferably performed over a period in a range from 0.5 to 4 hours and independently thereof at a temperature in the range from 80 to 150° C. Drying can be performed most simply by stopping heating the nitric acid in the storage vessel so that no further vapour is generated.

The carbon fibres can be positioned in the vapour stream in the reactor by means of a retaining device 12, for example. This retaining device can be a screen, grid or grate, for example.

In comparison to the conventional treatment with liquid nitric acid, the five-hour treatment with nitric acid vapour at 125° C., for example, appears to be an efficient method for using the carbon nanofibres as a support for catalysts, for example, which can be applied by impregnation.

In a further embodiment the object underlying the invention is achieved by carbon fibres which are characterised in that the ratio of oxygen atoms to carbon atoms derived from the atomic surface concentrations measured with XPS is greater than 0.18.

With the previously known methods it was not possible to produce carbon fibres with such a high surface concentration of oxygen. Surprisingly these carbon fibres have therefore been made available for the first time. In comparison to previously known surface-modified carbon fibres the carbon fibres according to the invention provide for the first time a material which opens up entirely new fields of application through further surface modification with organic molecules.

Such carbon fibres in which the ratio of oxygen atoms to carbon atoms, derived from the atomic surface concentrations measured with XPS, is greater than 0.2 are therefore particularly preferred. Within the meaning of the invention XPS stands for X-ray photoelectron spectroscopy.

For the subsequent use of the functionalised carbon nanofibres it is desirable for the functional groups generated at the surface of the carbon nanofibres in the nitric acid gas-phase treatment to be as reactive as possible for further subsequent reaction steps. Free unesterified carboxyl or carboxylic acid groups, which should be included in as high a number as possible, as well as carboxylic anhydride groups, which likewise have an adequate reactivity, are particularly reactive.

Surprisingly carbon fibres having in particular a particularly high proportion of carboxylic acid groups were obtainable for the first time through the use of the new oxidation method.

For that reason carbon fibres containing more than 400 μmol in total of carboxylic acid groups and carboxylic anhydride groups per g of carbon in chemically bonded form are also preferred. Such carbon fibres containing of this total more than 350 μmol of carboxylic acid groups per g of carbon in chemically bonded form are particularly preferred.

As low as possible an exit temperature in the TPD analysis is a reliable indication of as good a reactivity as possible of the functional group being eliminated for subsequent reactions. As CO₂ is predominantly eliminated at lower temperatures than CO, carbon nanofibres eliminating more than 45% of their chemically bonded oxygen in the TPD analysis as CO₂ are also preferred. Carbon fibres which contain more oxygen bonded in CO₂-eliminating or desorbing groups than in CO-eliminating groups are most particularly preferred.

In a further embodiment the object underlying the invention is achieved by carbon fibres obtainable by the method according to the invention.

In a yet further embodiment the object underlying the invention is achieved by the use of the carbon fibres according to the invention in composites, in energy stores, as sensors, as adsorbents, as supports for heterogeneous catalysts or as a catalytically active material.

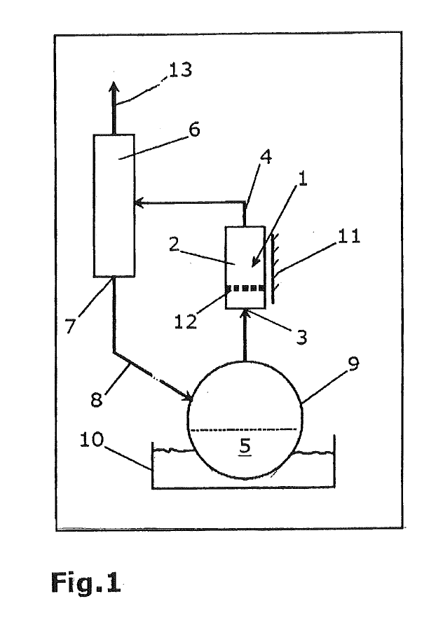

FIG. 1 shows a schematic view of the setup for the treatment of carbon nanofibres with nitric acid vapour. The multitube fixed-bed reactor is heated by means of a resistance heating tape, the round flask by means of an oil bath.

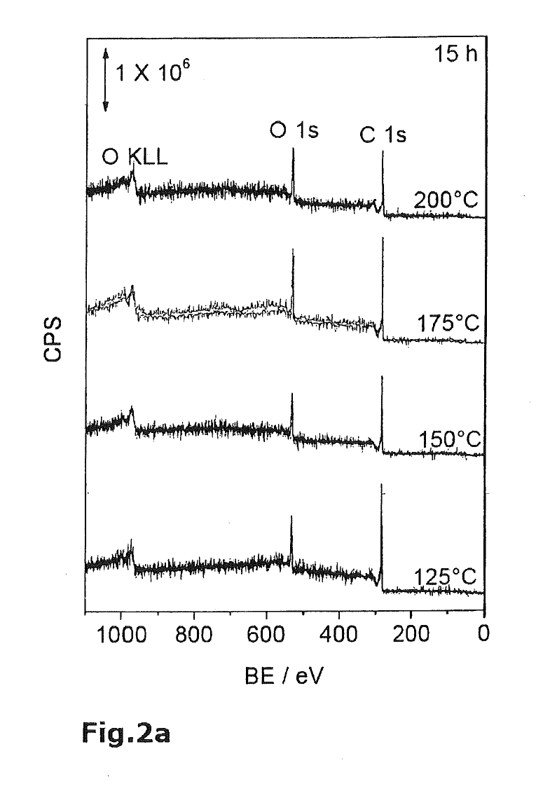

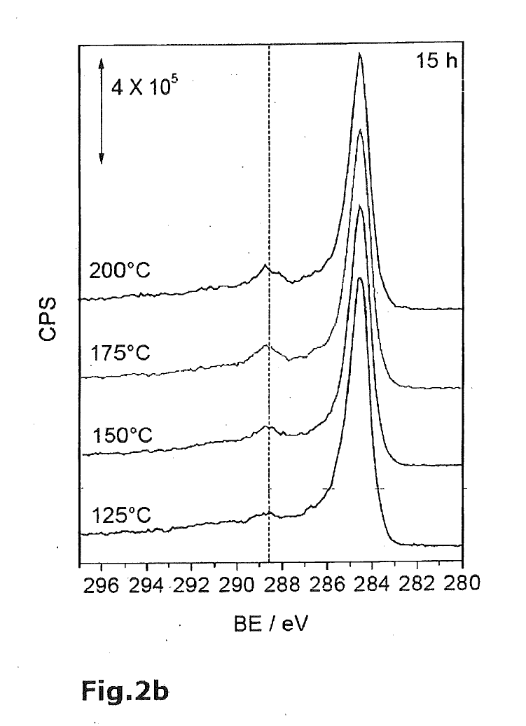

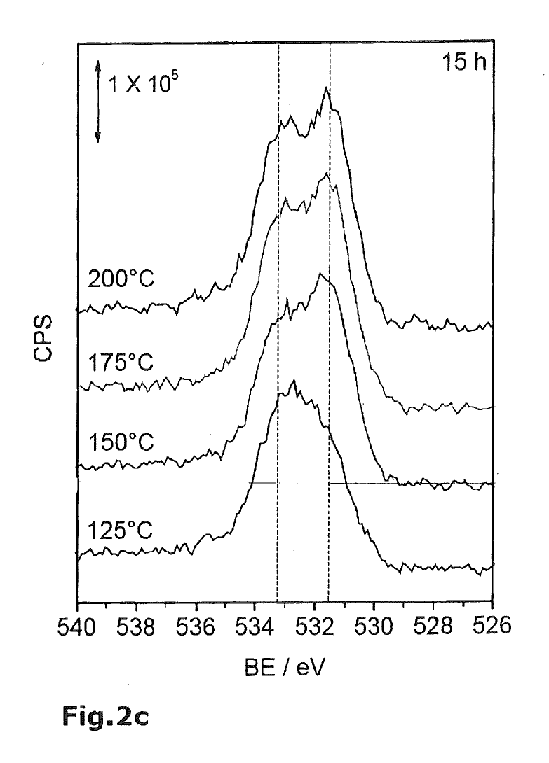

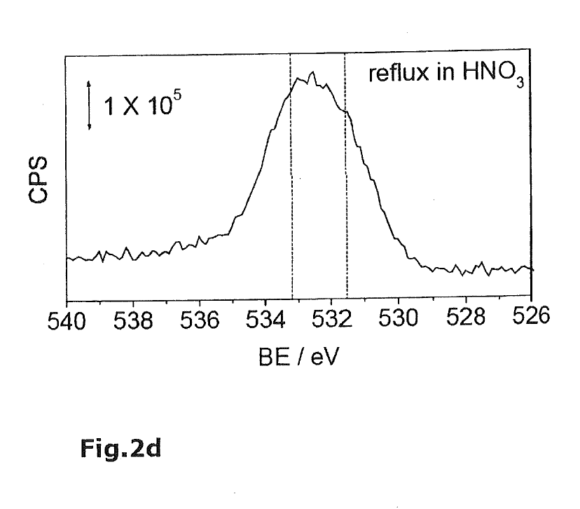

FIG. 2 shows the following XPS spectra: (a) XPS overview spectrum, (b) C 1s and (c) O 1s XP spectrum of carbon nanofibres which were treated for 15 hours with HNO₃ vapour at various temperatures. The O 1s spectrum of carbon nanofibres which were treated for 1.5 hours by means of the conventional method with liquid HNO₃ at 120° C. is shown in (d) for comparison.

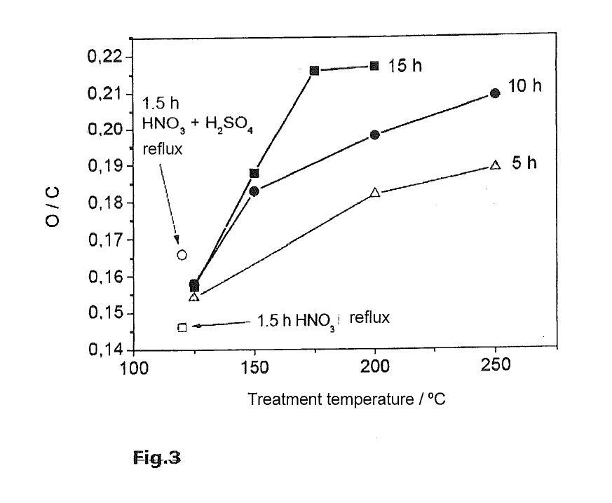

FIG. 3 shows the ratio of oxygen to carbon derived from the atomic surface concentrations (XPS) of carbon nanofibres which were treated with HNO₃ vapour for various times and at varying temperatures. The oxygen/carbon ratio after the conventional treatment is also shown for comparison.

FIG. 4 shows SEM images (a) of untreated carbon nanofibres and (b) of carbon nanofibres treated with HNO₃ vapour for 15 hours at 200° C.

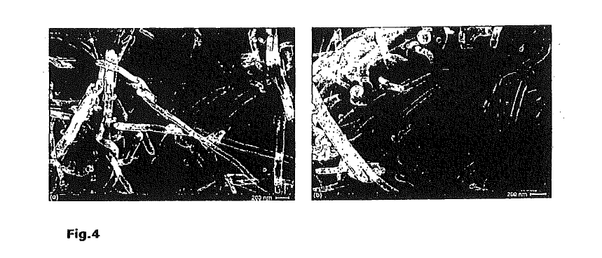

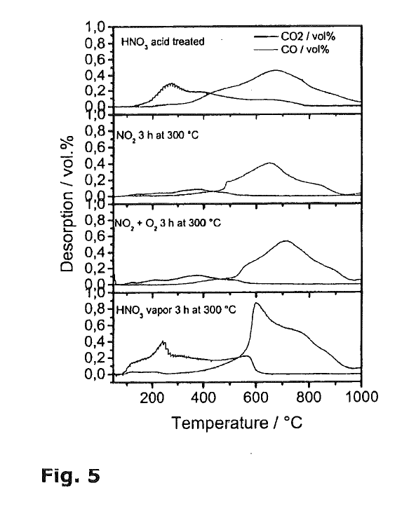

FIG. 5 shows the comparison of the TPD elimination profiles of carbon nanofibres when treated with gaseous HNO₃, NO₂, NO₂:O₂ (1:1) and liquid HNO₃. All treatments were performed for 3 hours. The graphs are all standardised to 1 g of carbon fibres.

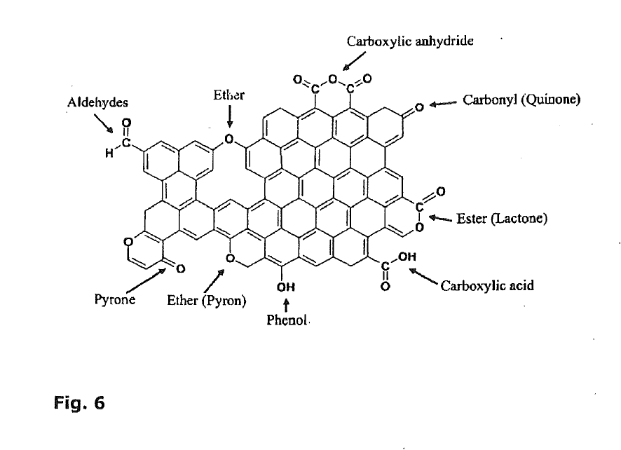

FIG. 6 shows an overview of the various chemically bonded oxygen-containing groups of carbon nanofibres.

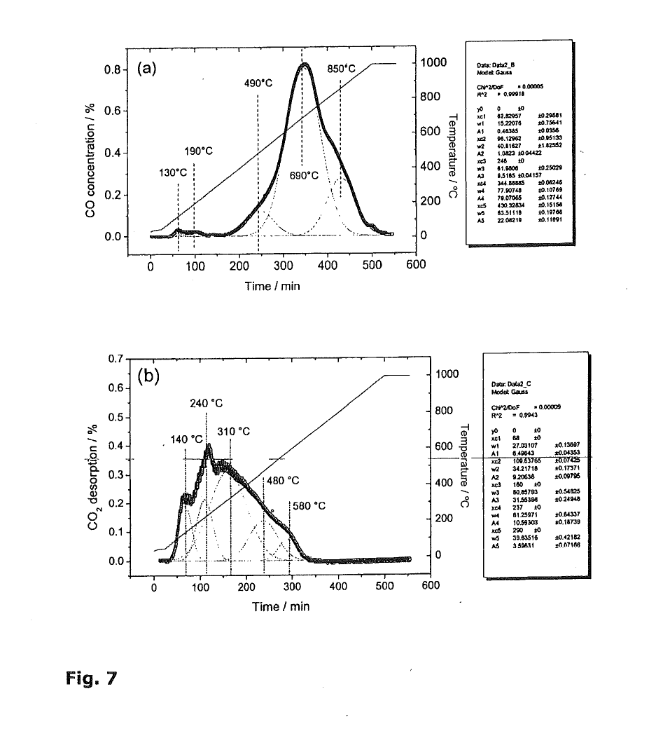

FIG. 7 shows the peak fittings method for the TPD profiles ((a) CO profile, (b) CO₂ profile) using the example of gas-phase treatment with HNO₃ at 200° C. for 15 hours.

Table 1 shows the values for quantification of the various functional groups from the TPD measurements for CO₂ elimination. The amounts are given in μmol/g (10⁻⁶ mol/g).

Table 2 shows the values for quantification of the various functional groups from the TPD measurements for CO elimination. The amounts are given in μmol/g (10⁻⁶ mol/g).

### EXAMPLES

The HNO₃ gas-phase treatment setup that was used is shown in FIG. 1. Typically 200 mg of carbon nanofibres 1 (50-200 nm diameter, Applied Sciences, Ohio, USA) were placed in the reactor 2 and in various experiments heated to a temperature of 125° C., 150° C., 175° C., 200° C., 250° C. The round flask 9 was filled with 150 ml of conc. HNO₃ 5 and heated to 125° C. whilst stirring. The countercurrent condenser 6 placed on top was connected to the exhaust gas. After a defined period of 5, 10 and 15 hours heating of the oil bath 10 was switched off and heating of the reactor 1 was maintained for a further 2 hours at 110° C. in order to dry the treated carbon nanofibres. Then the carbon nanofibres 1 were characterised extensively. The setup that was used effectively prevents the condensed liquid nitric acid within the condenser from flowing back across the sample. The treatment correspondingly took place entirely under gas phase conditions, as wetting of the carbon nanofibres with liquid nitric acid was completely avoided. The morphology of the carbon nanofibres was analysed by means of scanning electron microscopy (LEO Gemini 1530). X-ray photoelectron spectroscopy (XPS) was performed in an ultra-high vacuum plant using a Gammadata Scienta SES 2002 analyser. The pressure in the measuring chamber was 2×10⁻¹⁰ mbar. Al K₀ radiation (1486.6 eV; 14 kV; 55 mA) with a transmission energy of 200 eV was used as the X-ray radiation, allowing an energy resolution of better than 0.5 eV to be achieved. Possible charging effects were offset by the use of a source of slow electrons. The bonding energies were calibrated to the position of the main carbon signal (C 1s) at 284.5 eV.

XP spectroscopy is a proven method for characterising oxygen-containing functional groups. Different oxygen-containing groups can be distinguished using the C 1s and O 1s spectra (Okpalugo, T. I. T. et al., Carbon 43:153-61 (2005); Martinez, M. T. et al., Carbon 41:2247-56 (2003)). As an example the XP spectra are shown here for carbon nanofibres which were treated for 15 hours at various temperatures. FIG. 2(a) shows the XPS overview spectra of the carbon nanofibres after the 15-hour HNO₃ gas-phase treatment at various temperatures. The signals in the C 1s, O 1s and O KLL regions are clearly visible. The presence of nitrogen is indicated by a weak N 1s signal at approximately 400 eV. The intensity of the O 1s signal increases as the temperature rises, whereas that of the C 1s signal decreases correspondingly.

The assignment of signals in the C 1s region is carried out in the literature as follows (Lakshminarayanan, P. V. et al., Carbon 42:2433-42 (2004); Okpalugo, T. I. T. et al., Carbon 43:153-61 (2005)): carbon in graphite at 284.5 eV, carbon singly bonded to oxygen in phenols and ethers (C—O) at 286.1 eV, carbon doubly bonded to oxygen in ketones and quinones (C═O) at 287.5 eV, carbon bonded to two oxygen atoms in carboxyl groups, carboxylic anhydrides and esters (—COO) at 288.7 eV and the characteristic “shake-up” line of carbon in aromatic compounds at 190.5 eV (n→n*transitions). The C 1s spectrum after a 15-hour HNO₃ gas-phase treatment is shown in FIG. 2(b). The increasing size of the shoulder as the temperature rises at higher bonding energies of the C is main signal at 284.5 eV can be seen by comparing the signal symmetry. The strong growth of the signal at 288.7 eV, signalling a sharp rise in the amount of —COO groups, is even clearer. These are mainly carboxyl groups and anhydrides, which are among the most important oxygen-containing functional groups on carbon surfaces for various applications.

The O 1s core level spectrum of the same batch of treated carbon fibres is shown in FIG. 2(c). The two main contributions are shown by the dotted lines and are assigned respectively to the oxygen atoms (C═O) doubly bonded to carbon in quinones, ketones or aldehydes at 531.5 eV and to the oxygen atoms (C—O) singly bonded to carbon in ethers, hydroxyl groups or phenols at 533.2 eV (Bubert, H. et al., Anal. Bioanal. Chem. 374:1237-41 (2002); Zhang, J. et al., J. Phys. Chem. B 107:3712-8 (2003)). As both singly and doubly carbon-bonded oxygen atoms occur in esters, carboxyl groups, anhydrides or pyrans, both oxygen atoms of these groups contribute to the two O 1s signals. In the O 1s spectra it is clear that at relatively low treatment temperatures the main signal is dominated by the C—O single bond, which is presumably attributable to the preferred formation of hydroxyl groups at low temperatures. As the temperature increases, the formation of C═O double bonds rises sharply. For the purposes of comparison the O 1s spectrum of carbon nanofibres with conventional HNO₃ treatment is shown in FIG. 2(d). Here the contribution to the signal at 533.2 eV is greater than at 531.6 eV and is similar to the spectrum for HNO₃ gas-phase treatment at low temperatures. Results showing a similar trend have been obtained in the literature with the conventional wet HNO₃ method, i.e. the signal at 533.2 eV was greater than that at 531.6 eV (Martinez, M. T. et al., Carbon 41:2247-56 (2003)). Thus HNO₃ gas-phase treatment not only improves the yield but also changes the number of different oxygen-containing functional groups on the carbon nanofibres as compared with the conventional method with liquid HNO₃. It is known that the formation of different oxygen species, such as e.g. C═O, is extremely dependent on temperature. Owing to the azeotropic boiling point limit of concentrated HNO₃ of 122° C. it is not possible to perform conventional HNO₃ treatment at temperatures above 122° C. and atmospheric pressure, as a result of which the production of certain species within a predefined reaction time is limited.

The atomic surface concentrations of carbon and oxygen were determined by means of XPS measurements (Ma, W. et al., Catal. Today 102-103:34-9 (2005)). The ratio of oxygen to carbon (O/C) in the carbon nanofibres after various treatments is shown in FIG. 3. It can be seen that the O/C ratio after an HNO₃ treatment at 125° C. is around 0.155, which is somewhat higher than with a conventional HNO₃ treatment at 120° C. for 1.5 hours and somewhat lower than with a conventional mixed acid treatment (HNO₃ and H₂SO₄) at 120° C. for 1.5 hours. The ratio increases as the temperature rises and the treatment period lengthens. After 15 hours of treatment at 175° C. or 200° C. the ratio is more than 0.21. Under these conditions the amount of oxygen on the carbon nanofibres appears to reach the saturation limit, as shown by the flattening of the correlation curve.

Following HNO₃ gas-phase treatment the carbon nanofibres were able to be used in further processes with no additional processing steps such as filtration, washing or drying, for example. No change in the bulk density of the carbon nanofibres was observed after treatment, and the SEM images confirm that no morphological changes to the carbon nanofibres occurred as a result of the treatment (FIG. 4). The commonly occurring agglomeration caused by conventional treatment with liquid HNO₃ was not observed with HNO₃ gas-phase treatment. Furthermore, the morphology of the carbon nanofibres is not changed by the gas-phase treatment (FIG. 3). The treatment of carbon nanofibres grown on various carbon substrates such as graphite film or carbon fibres was also compared (Briggs, D. et al., John Wiley & Sons 635-6 (2004); Li, N. et al., Adv. Mater. 19:2957-60 (2007)). After refluxing for 1.5 hours in a stirred HNO₃ solution the carbon nanofibres had largely become detached from the substrate, resulting in a dark-coloured suspension. After HNO₃ gas-phase treatment, however, the carbon nanofibres remained intact on the substrate. This result is particularly important for carbon nanofibre applications in which the secondary structure needs to be maintained, for example in vertically oriented carbon nanofibres or branched carbon nanofibre composites.

In order to obtain information about the nature of the functional groups reacted on the carbon nanofibres, TPD (temperature-programmed desorption) measurements were performed.

To this end approx. 150 to 200 mg of the functionalised carbon nanofibres (Baytubes C150P, treated with HNO₃ gas for 3 hours at 300° C.) were placed in a horizontal quartz tube with a 10 mm internal diameter and helium (99.9999% purity, flow rate 30 sccm) was passed over as the carrier gas. The sample was then heated from room temperature to 1000° C. at a heating rate of 2 K/min and the released amounts of CO and CO₂ were determined using an online infrared detector (Binos) in the gas stream. The temperature was held at 1000° C. for a total of one hour before the sample was cooled back down to room temperature. The detector itself was first calibrated with the specified gases for a measuring range of 0 to 4000 ppm.

For the purposes of comparison with other methods of oxidative functionalisation, carbon nanofibres (Baytubes C150P) were treated conventionally in the liquid phase with HNO₃ and also in the gas phase with NO₂ and with a mixture of NO₂ and O₂. These gas-phase treatments were performed in a vertical quartz tube with an internal diameter of 20 mm. In one experiment NO₂ (10 vol. % in helium) was passed through the bed of carbon nanofibres at a flow rate of 10 sccm. For the treatment with NO₂+O₂, oxygen (20.5 vol. % in N₂, 5 sccm) was additionally passed through in the NO₂/He gas stream in order to establish an NO₂:O₂ ratio of 1:1 in the carrier gas. For the treatment in the liquid phase the carbon nanofibres were refluxed for 3 hours in concentrated nitric acid (65%, J. T. Baker).

The results (FIG. 5) show a markedly different release of CO and CO₂ as a function of temperature for the differently functionalised carbon nanofibres. It clearly follows from this that the carbon nanofibres treated with HNO₃ in the gas phase release larger amounts of both CO and CO₂, indicating overall a higher surface functionalisation with oxygen-containing groups. In addition, the sample treated with HNO₃ in the gas phase shows a high release rate of both CO and CO₂ at approx. 600° C., indicating in particular a high proportion of carboxylic anhydride functionalities.

However, the release curves in FIG. 5 also show that CO is released at very much higher temperatures than CO₂. This is due to the higher bonding strength of the functional groups from which CO is eliminated. FIG. 6 provides an overview of the functional groups usually present in oxidised carbon nanofibres. The following assignment for elimination temperatures can be taken from the literature:

| CO₂: | chemisorbed CO₂ | below 250° | C. |
|---|---|---|---|
| | carboxylic acid | 310° | C. |
| | carboxylic anhydride | 420° | C. |
| | lactone | 580° | C. |
| CO: | aldehyde, ketone | below 300° | C. |
| | carboxylic anhydride | 420° | C. |
| | phenol, ether | 700° | C. |
| | pyrone | 830° | C. |

Based on these assignments, a sum of curves with Gaussian normal distribution was adjusted to the TPD curves (FIG. 7), and from this the quantitative assignment (Tables 1 and 2) to the functional groups originally contained in the carbon nanofibres was determined.

TABLE 1

| | CO₂ | Carboxylic | Carboxylic | |
|---|---|---|---|---|
| Sample | chemisorbed | acid | anhydride | Lactone |
| 15 h at 200° C. | 87 | 546 | 142 | 47 |
| HNO₃ liquid | 118 | 305 | 58 | 46 |
| NO₂ gas, 3 h at | 8 | 131 | 24 | 0 |
| 200° C. | | | | |

TABLE 2

| | Ketone, | Carboxylic | Phenol, | |
|---|---|---|---|---|
| Sample | aldehyde | anhydride | ether | Pyrone |
| 15 h at 200° C. | 28 | 142 | 1023 | 317 |
| HNO₃ liquid | 58 | 105 | 741 | 197 |
| NO₂ gas, 3 h at | 12 | 35 | 250 | 87 |
| 200° C. | | | | |

1.-14. (canceled)

15. A method for the functionalisation of carbon fibres, wherein
a) placing carbon fibres in a reactor, which has an inlet and an outlet,
b) heating the reactor to a temperature in a range from 125 to 500° C.,
c) passing vapour from nitric acid through the reactor, and subsequently
d) drying the treated carbon fibres.

16. The method according to claim 15, further comprising using carbon nanofibres having an external diameter in a range from 3 to 500 nm as the carbon fibres.

17. The method according to claim 15, wherein the carbon fibers placed in the reactor have a BET surface area ranging from 10 to 500 m²/g,

18. The method according to claim 17, wherein the BET surface area ranges from 20 to 200 m²/g.

19. The method according to claim 15, further comprising connecting a condenser to the reactor outlet, wherein a condenser outlet for a condensate is connected via a return line to a storage vessel for the nitric acid.

20. The method according to claim 19, further comprising using a glass flask as a storage vessel for the nitric acid, and heating the nitric acid with an oil bath.

21. The method according to claim 15, wherein after step b), holding the reactor at the temperature for a period ranging from 3 to 20 hours,

22. The method according to claim 21, wherein the period ranges from 5 to 15 hours.

23. The method according to claim 15, further comprising performing step c) over a period in a range from 0.5 to 4 hours and independently thereof at a temperature in a range from 80 to 150° C.

24. The method according to claim 15, wherein the treated and dried carbon fibres have a ratio of oxygen atoms to carbon atoms derived from atomic surface concentrations, as measured by XPS, of greater than 0.18

25. Carbon fibres, wherein a ratio of oxygen atoms to carbon atoms derived from atomic surface concentrations, as measured by XPS, is greater than 0.18.

26. The carbon fibres according to claim 25, wherein the fibres have an average diameter of 3 to 500 nm and a ratio of length to diameter of at least 5:1.

27. Carbon fibres, wherein the fibres contain more than 350 μmol of carboxylic acid groups per g of carbon in a chemically bonded form.

28. The carbon fibres according to claim 27, wherein the fibres contain more than 400 μmol in total of carboxylic acid groups and carboxylic anhydride groups per g of carbon in a chemically bonded form.

29. The carbon fibres according to claim 27, wherein the fibers eliminate more than 45% of chemically bonded oxygen in a TPD analysis as CO2.
